(19) United States
(12) Patent Application Publication
(10) Pub. No.: US 2015/0368213 A1
(43) Pub. Date: Dec. 24, 2015

(54) Novel Inhibitors of System Xc-

(71) Applicant: The University of Montana, Missoula, MT, Missoula, MT (US)

(72) Inventors: Nicholas R. Natale, Missoula, MT (US); Richard J. Bridges, Missoula, MT (US); Sarjubhai A. Patel, Missoula, MT (US); Nathan S. Duncan, Reeds Spring, MO (US); Matthew J. Weaver, Missoula, MT (US); Mariusz P. Gajewski, Russellville, AR (US)

(21) Appl. No.: 14/744,707

(22) Filed: Jun. 19, 2015

Related U.S. Application Data
(60) Provisional application No. 62/015,178, filed on Jun. 20, 2014.

(51) Int. Cl. C07D 261/08; C07C 311/42; A61K 49/00; C07D 261/18; A61K 51/04

(52) U.S. Cl. CPC C07D 261/08; C07D 261/18; A61K 51/0453; A61K 49/0052; C07C 311/42; A61K 51/0497

(57) ABSTRACT

The present invention is directed to novel inhibitors of system X_{c}⁻, also known as the cystine/glutamate antiporter. The present invention is further directed to methods of treating and detecting cancer that overexpresses system X_{c}⁻ which includes but is not limited to gliomas (including glioblastoma), triple negative breast cancer, and bladder cancer. The present invention is further directed to methods of treating seizures including epileptic seizures.

### BACKGROUND OF THE INVENTION

System x_{c}⁻, also known as the glutamate/cystine antiporter, (“Sx_{c}⁻”) is a transmembrane protein expressed in a variety of cells, which include neural (e.g. astrocytes, microglia, immature cortical neurons and glioma cells) and non-neural (e.g. fibroblasts, macrophages, hepatocytes and endothelial) cells. Sx_{c}⁻ functions as an antiporter/exchanger to import L-cystine into the cell and export L-glutamate out of the cell. The imported L-cystine is essential within the cell for the production of the body's primary antioxidant, glutathione (“GSH”), and the exported L-glutatmate can act as an extracellular neutrotransmitter. Due to its bimodality, Sx_{c}⁻ has been linked to a wide range of central nervous system (“CNS”) functions, including oxidative protection, the operation of the blood—brain barrier, neurotransmitter release, synaptic organization and cyto-architecture, viral pathology, drug addiction, chemosensitivity, chemoresistance, and tumor growth within the brain as well as in peripheral compartments (e.g., breast and bladder).

Glioblastoma multiforme (“GBM”) is an aggressive and malignant brain tumor that arises from glial cells in the brain. GBM is a deadly form of cancer with a median survival rate of 4.5 months without treatment and about 13 months with aggressive treatment. Almost all patients diagnosed with GBM die within 5 years. Glial cells express an abundance of Sx_{c}⁻. The import of L-cystine by Sx_{c}⁻ leads to production of GSH which in high intracellular levels in cancer cells is associated with resistance to drugs such as temozolomide (TMZ), the chemotherapeutic agent of choice for GBM. The export of L-glutamate through Sx_{c}⁻ from glioma cells is associated with peritumoral seizures and acts to destroy surrounding neurons allowing the tumor to grow. Thus, Sx_{c}⁻ is a drug target that is uniquely well-suited to provide therapeutic benefit in GBM as well as potentially other cancer indications where Sx_{c}⁻ is overexpressed (e.g., triple negative breast cancer).

Seizures refer to the involuntary and repeated contracting and relaxing of the subject's muscles caused by excessive release of neurotransmitters. Seizures have many causes including both genetic and environmental factors including epilepsy, brain tumors and infection. Approximately 1 in 10 people will suffer a seizure in their lifetime. In the United States, over 3 million people suffer from epileptic seizures and 50,000 of those affected die each year from seizures and related causes. Seizures, including epileptic seizures and chronic seizure states such as status epilepticus, involve the activation of AMPA and NMDA glutamate receptors. In fact, increase in glutamate release has been found in chronic epilepsy models in rodents. Rowley N.M. et al. Glutamate and GABA synthesis, release, transport and metabolism as targets for seizure control Neurochem Int. 2012 Sep, 61(4), 546-58.

Current inhibitors of Sx_{c}⁻ include L-α-aminoadipate, L-α-aminopimelate, L-homocysteate, L-ser-O-sulphate, L-β-N-oxalyl-L-α,β-diaminopropionate, L-alanosine, quisqualate, ibotenate, (RS)-4-Br-homoibotenate, S-2-naphthyl-ethyl-amino-3-carboxy-methyl isoxazole propionic acid, bis-trifluoromethylphenyl-isoxazole-4hydrazone, 5-naphthylethylisoxazole-4-(2,4-dinitrophenol) hydrazone-dinitrophenol), (S)-4-carboxyphenylglycine, sulphasalazine, and sulphonic acid phenyglycine. Despite the discovery of several Sx_{c}⁻ inhibitors none have both the selectivity and affinity to treat the myriad of diseases associated with over-expression and/or over-activation of Sx_{c}⁻.

Thus, because of their potentially broad therapeutic utility for a variety of devastating disorders with unmet need, there is a need in the art for compounds that exhibit both selectivity and potent inhibition of Sx_{c}⁻. One mechanism to affect such results is to develop compounds that act as inhibitors of Sx_{c}⁻.

### SUMMARY OF THE INVENTION

The present invention provides inhibitors of Sx⁻.

In one embodiment, the present invention provides a compound of formula A-B-D wherein:
A is selected from —C—OH, —COOH, a compound of formula (I)

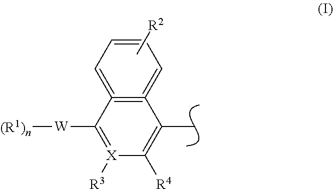

and formula

(II)

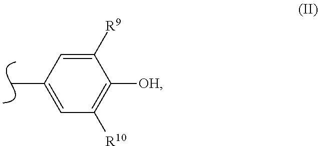

wherein:
n is an integer of 0 or 1;
W, R⁹ and R¹⁰ are each independently selected from an H, a halogen, a nitrile, a carbonyl and a nitro group;
X is selected from C, N, O, P, and S;
R¹ and R² are each independently selected from H, dimethylamine, an optionally substituted alkyl, an optionally substituted aryl and an optionally substituted heteroaryl; and R³ and R⁴ are each independently selected from, H, O, —O—CH₃, —O—CH₂-aryl, and a C₁-C₆ alkyl, wherein R³ and R⁴ taken together with the atoms to which they are attached optionally form a 6-membered cycloalkyl or heterocycloalkyl,
   B is a linker compound selected from , and

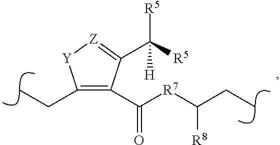

and

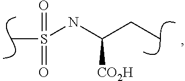

wherein:
Y is C or N;
Z is C or O;
each R⁵ is independently selected from H, an optionally substituted C₂-C₆ alkyl, an optionally substituted aryl, an optionally substituted heteroaryl and

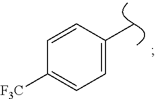

R⁷ is selected from —N—, —NH₂—C₁—C₆ alkyl-NH₂—, and piperazine; and
R⁸ is selected from —CO₂H, and —CO₂—CH₂—CH₃, and
D is a compound of formula (II) wherein R⁹ and R¹⁰ are each independently selected from an H, a halogen, a nitrile, a carbonyl and a nitro group,
or a pharmaceutically acceptable salt, ester or prodrug thereof.

In a preferred embodiment, the present invention provides a compound of formula A-B-D wherein A is a compound of formula (I), B is

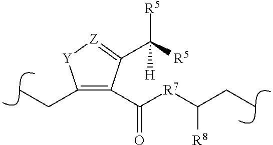

and D is compound of formula (II), or a pharmaceutically acceptable salt, ester or prodrug thereof.

In a more preferred embodiment, the present invention provides a compound of formula (III),

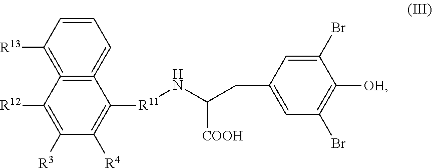

wherein:
R³ and R⁴ are each independently selected from, H, O—CH₃, O—CH₂-phenyl, and a C₆ alkyl, wherein R³ and R⁴ taken together with the atoms to which they are attached optionally form a 6-membered cycloalkyl;

R¹¹ is selected from

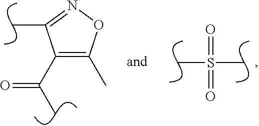

R¹² is selected from H and Cl; and
R¹³ is selected from H and dimethylamine.

In another embodiment, the present invention provides a compound of formula (IV),

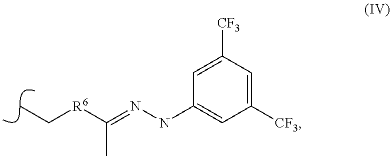

wherein:

R⁶ is selected from

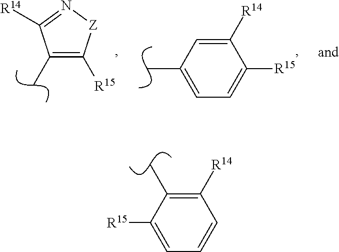

wherein Y is C or N and Z is C or O;

R¹⁴ is selected from —COOH, —C—OH, and and

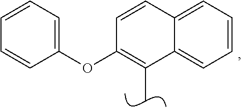 [SMILES: Cc1c(Oc2ccccc2)ccc2ccccc12]

and

R¹⁵ is selected from methyl,

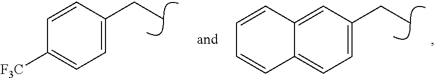

wherein if R¹⁵ is methyl then R¹⁴ is not —COOH or —C—OH.

In another embodiment, the present invention provides a composition comprising a compound of the present invention and one or more pharmaceutically acceptable carriers.

In yet another embodiment, the present invention provides a method of treating a disease or disorder selected from a glioma, a seizure, schizophrenia, Parkinson's, and a viral infection of the brain comprising administering to a person in need thereof a therapeutically effective amount of a compound of the present invention.

In a preferred embodiment, the present invention provides a method of treating a tumor comprising administering to a subject in need thereof a therapeutically effective amount of a compound of the present invention.

In a preferred embodiment, the present invention provides a method of treating a tumor expressing abnormally elevated levels of Sx_{c}⁻ comprising administering to a subject in need thereof a therapeutically effective amount of a compound of the present invention.

In a preferred embodiment, the present invention provides a method of treating a tumor comprising administering to a subject in need thereof a therapeutically effective amount of a compound of the present invention, wherein the tumor expresses a greater amount of Sx_{c}⁻ than surrounding tissue.

In a preferred embodiment, the present invention provides a method of treating a tumor comprising administering to a subject in need thereof a therapeutically effective amount of a compound of the present invention, wherein the tumor expresses a higher level of Sx_{c}⁻ than surrounding tissue.

In a more preferred embodiment, the present invention provides a method of treating a glioma, preferably a glioblastoma, more preferably glioblastoma multiforme, comprising administering to a subject in need thereof a therapeutically effective amount of a compound of the present invention.

In another preferred embodiment, the present invention provides a method of treating a seizure, preferably an epileptic seizure, more preferably status epilepticus comprising administering to a subject in need thereof a therapeutically effective amount of a compound of the present invention.

In another embodiment, the present invention provides a method for detecting cancer in vivo, comprising:
(i) administering to a patient in need thereof a diagnostically effective amount of a compound of the present invention wherein at least one atom is an atom selected from carbon-11 (11C), fluorine-18 (18F), nitrogen-13 (13N), oxygen-15 (15O) or a combination thereof;
(ii) detecting whether a tissue suspected of having cancer in the patient retains a higher level of the compound of (i) than surrounding tissue,
   wherein a higher retention level of the compound of (i) indicates cancer and wherein the detection is carried out by positron emission tomography (PET) scanning

In another embodiment, the present invention provides a method for detecting cancer in vivo, comprising:
(i) administering to a patient in need thereof a diagnostically effective amount of a compound of the present invention;
(ii) detecting whether a tissue suspected of having cancer in the patient retains a higher level of the compound of (i) than surrounding tissue,
   wherein a higher retention level of the compound of (i) indicates cancer and wherein the detection is carried out by fluorescent imaging.

### BRIEF DESCRIPTION OF THE DRAWINGS

FIG. 1-Michaelis-Menten analysis of 5-4-TFM-Benzyl-4-bis-TFM-HMICA binding rate kinetics.

FIG. 2-LWB replot of the Michaelis-Menten analysis of 5-4-TFM-Benzyl-4-bis-TFM-HMICA.

FIG. 3-LWB slope vs. [I] replot of the Michaelis-Menten analysis of 5-4-TFM-Benzyl-4-bis-TFM-HMICA.

FIG. 4-Replot of Eadie-Hofstee analysis of 5-4-TFM-Benzyl-4-bis-TFM-HMICA binding rate kinetics.

### DETAILED DESCRIPTION OF THE INVENTION

The glutamate/cystine antiporter (“Sx_{c}⁻”) directly binds glutamate and cystine to transport them across the plasma membrane. Mimics of these compounds were previously developed as competitive inhibitors of Sx_{c}⁻. Bridges et al., System xc-cystine/glutamate antiporter: an update on molecular pharmacology and roles within the CNS, Br J Pharmacol, 2012 January 165(1), 20-34. It is a discovery of the present invention that Sx_{c}⁻ can also be inhibited via allosteric sites. Specifically, it is a discovery of the present invention that the substrate binding domain of Sx_{c}⁻ is flanked by lipophilic domains which act as allosteric sites. Compounds of the present invention may act:

(1) entirely via these allosteric sites as non-competitive inhibitors;

(2) entirely via the substrate binding sites as competitive inhibitors; or

(3) via both the allosteric sites and substrate binding sites as mixed inhibitors.

### Definitions

As used herein, the term “treating” includes preventative as well as disorder remittent treatment including reducing, suppressing and inhibiting disease progression or recurrence. As used herein, the terms “reducing”, “suppressing” and “inhibiting” have their commonly understood meaning of lessening or decreasing. As used herein, the term “progression” means increasing in scope or severity, advancing, continuing, growing or becoming worse. As used herein, the terms “recurrence” and “recurrent” refer to the return of a disease after a remission.

As used herein, the term “administering” refers to bringing a patient, tissue, organ or cells in contact with a compound of the present invention. As used herein, administration can be accomplished in vitro (i.e. in a test tube) or in vivo, (i.e. in cells or tissues of living organisms, for example, humans).

As used herein, the term “effective amount” refers to an amount sufficient to affect a desired biological effect, such as a beneficial result, including, without limitation, prevention, diminution, amelioration or elimination of signs or symptoms of a disease or disorder or an amount sufficient to aid in detection Thus, the total amount of each active component of the pharmaceutical composition or method is sufficient to show a meaningful subject benefit. Thus, an “effective amount” will depend upon the context in which it is being administered. An effective amount may be administered in one or more prophylactic, therapeutic or diagnostic administrations.

As used herein the term “therapeutically effective amount” refers to that amount of the compound being administered sufficient to prevent development of, alleviate to some extent one or more of the symptoms, or the signs of the condition or disorder being treated. The “therapeutically effective amount” can vary depending on the compound, the disease or disorder and its severity, and the age, weight, etc., of the subject to be treated.

As used herein the term “diagnostically effective amount” refers to that amount of the compound being administered sufficient to provide detection of the presence of the compound by standard medical diagnostic means such as PET imaging and fluorescent imaging.

As used herein the term “linker compound” refers to any chemical compound or compounds capable of forming a chemical bond with two or more other distinct chemical compounds such that all compounds form a single larger compound. In one embodiment, the linker compound is a bond. Multiple linker compounds may be used in the formation of the larger compound.

As used herein, the term “composition” is intended to encompass a product comprising the specified ingredients in the specified amounts, as well as any product which results, directly or indirectly, from a combination of the specified ingredients in the specified amounts.

As used herein the term “subject” refers to animals such as mammals, including but not limited to, primates (e.g., humans), cows, sheep, goats, horses, dogs, cats, rabbits, rats, mice and the like. In preferred embodiments, the subject is a human.

As used herein the term “tumor” refers to both benign and malignant tumors and includes all cancer types.

The term “prodrug” or “prodrugs” refers to compounds, including monomers and dimers of the compounds of the invention, which have cleavable groups and become under physiological conditions compounds which are pharmaceutically active in vivo.

As used herein “ester” or “esters” is represented by the formula —OC(O)A¹ or —C(O)OA¹, where A^{l} can be alkyl, cycloalkyl, alkenyl, cycloalkenyl, alkynyl, cycloalkynyl, aryl, a heteroaryl group or other suitable substituent.

As used herein, the term “pharmaceutically acceptable” describes a material that is not biologically or otherwise undesirable, i.e., without causing an unacceptable level of undesirable biological effects or interacting in a deleterious manner.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), carbon-14 (¹⁴C), carbon-11 (¹¹C), oxygen-15 (¹⁵O), nitrogen-13 (¹³N), and fluorine-18 (¹⁸F). All isotopic variations of the compounds of the present invention, whether radioactive or not, are intended to be encompassed within the scope of the present invention.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as ChemDraw™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

As used herein, the terms “optional” or “optionally” means that the subsequently described event or circumstance can or cannot occur, and that the description includes instances where said event or circumstance occurs and instances where it does not.

As used herein, the term “substituted” is contemplated to include all permissible substituents of organic compounds. In a broad embodiment, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain embodiments, unless expressly indicated to the contrary, individual substituents can be further optionally substituted (i.e., further substituted or unsubstituted).

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain embodiments, unless expressly indicated to the contrary, individual substituents can be further optionally substituted (i.e., further substituted or unsubstituted).

Suitable monovalent substituents on a substitutable carbon atom of an “optionally substituted” group are each independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄0(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o})C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂;—N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄ SSR^{o}; —(C₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; –P(O)₂R^{o}; —P(O)R^{o}₂; —OP(O)R^{o}₂; —OP(O)(OR^{o})₂; SiR^{o}₃; —(C₁₋₄ straight or branched)alkylene)O—N(R^{o}; —(C₁₋₄ straight or branched)alkylene)C(O)O—N(R^{o})₂, wherein each R^{o}may be substituted as defined below and is independently hydrogen, C₁₋₆ aliphatic, —CH₂Ph, —O(CH₂)₀₋₁Ph, —CH₂-(5-6 membered heteroaryl ring), or a 5-6-embered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{•} (or the ring formed by taking two independent occurrences of R^{•} together with their intervening atoms), are independently halogen, —(CH₂)₀₋₂R^{•}, -(haloR^{•}), —(CH₂)₀₋₂OH, —(CH2)₀₋₂OR^{•}, —(CH₂)₀₋₂CH(OR^{•})₂; O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}3, —OSiR^{•}3, C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C1-4 aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —(C(R*₂))₂₋₃O—, or —S(C(R*₂))₂₋₃S—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*2)2-3O—, wherein each independent occurrence of R* is selected from hydrogen, C1-6 aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, (haloR^{•}), OH, —OR^{•}, —O(haloR^{•}), —CN, —C(O)OH, —C(O)OR^{•}, —NH2, —NHR^{•}, —NR^{•}2, or —NO₂, wherein each R^{•} is unsubstituted or where preceded by “halo” is substituted only with one or more halogens, and is independently C1-4 aliphatic, —CH₂Ph, —O(CH₂)0-1Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R†, —NR†₂, —C(O)R†, —C(O)OR†, —C(O)C(O)R†, —C(O)CH₂C(O)R†, —S(O)₂R†, S(O)₂NR†₂, —C(S)NR†₂, —C(NH)NR†2, or —N(R†)S(O)₂R†; wherein each R† is independently hydrogen, C1-6 aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R†, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R† are independently halogen, —R^{†}, (haloR^{†}), —OH, —OR^{†.}, —O(haloR^{†}), —CN, —C(O)OH, —C(O)OR^{†}, —NH₂, —NHR^{†}, —NR^{†}₂, or NO2, wherein each R^{†} is unsubstituted or where preceded by “halo” is substituted only with one or more halogens, and is independently C₁ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as 2 H, 3 H, 13 C, 14 C, 15 N, 18 O, 17 O, 35 S, 18F and 36 Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as 3 H and 14 C are incorporated, are useful in drug and/or substrate tissue distribution assays. Tritiated, i.e., 3 H, and carbon-14, i.e., 14 C, isotopes are particularly preferred for their ease of preparation and detectability. Further, substitution with heavier isotopes such as deuterium, i.e., 2 H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labeled reagent.

It is also appreciated that certain compounds described herein can be present as an equilibrium of tautomers. For example, ketones with an a-hydrogen can exist in an equilibrium of the keto form and the enol form.

Likewise, amides with an N-hydrogen can exist in an equilibrium of the amide form and the imidic acid form. Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

As used herein “n” is an integer of 0 or 1.

The term “alkyl” as used herein is a branched or straight-chain alkyl consisting of a saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or straight-chained. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, thiol, a phosphate or a sulfate.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, nitrile, sulfonamide, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

As used herein the term “carbonyl” refers to the formula —C═O.

As used herein the term “halogen” refers to an element selected from fluorine, chlorine, bromine, iodine and astatine.

As used herein the term “nitro group” refers to the formula —NO₂.

As used herein the term “nitrile” refers to the formula —C≡N.

As used herein the term “piperazine” refers to the formula

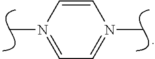

“C”, “H”, “N”, “O”, “P”, and “S”, as used herein, refer to the elements carbon, hydrogen, nitrogen, oxygen, phosphorous and sulfur, respectively.

As used herein the term “cycloalkyl” refers to a C₁-C₂₄ alkyl in the form of one or more rings.

As used herein the term “heterocycloalkyl” refers to a C₁-C₂₄ alkyl in the form of one or more rings wherein one or more carbons are each independently substituted with a C, N, O, P, or S.

“W”, as used herein, refers to an element or compound selected from H, a halogen, a nitrile, a carbonyl and a nitro group.

“X”, as used herein, refers to an element selected from C, N, O, P, and S.

“Y”, as used herein, refers to an element selected from C and N.

“Z”, as used herein, refers to an element selected from C and O.

“R¹” and “R²”, as used herein, each independently refer to H, dimethylamine, an optionally substituted alkyl, an optionally substituted aryl and an optionally substituted heteroaryl.

“R³” and “R⁴”, as used herein, each independently refer to an element or a compound selected from H, O, —O—CH3, —O—CH₂-aryl, and a C₁-C₆ alkyl. Optionally R³ and R⁴ taken together with the atoms to which they are attached form a 6-membered cycloalkyl or heterocycloalkyl.

“R⁵”, as used herein, refers to a compound selected from H, an optionally substituted C₂-C₆ alkyl, an optionally substituted aryl, an optionally substituted heteroaryl and

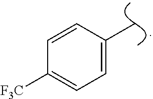

“R⁶”, as used herein, refers to a compound selected from

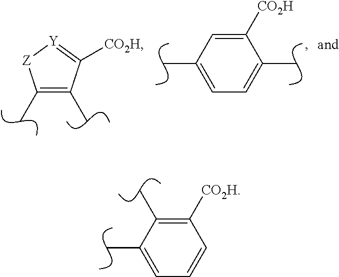

“R⁷”, as used herein, refers to an element or a compound selected from —N—, —NH₂—C₁-C₆ alkyl-NH₂—, and piperazine.

“R⁸”, as used herein, refers to a compound selected from —CO₂H, and —CO₂—CH₂—CH₃,

“R⁹” and “R¹⁰” as used herein, are each independently selected from an H, a halogen, a nitrile, a carbonyl and a nitro group.

“R¹¹” as used herein, refers to a compound selected from

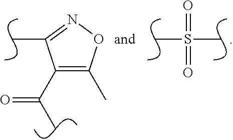

“R¹²” as used herein, refers to a compound selected from H and Cl, and

“R¹³” as used herein, refers to a compound selected from H and dimethylamine.

“R¹⁴” as used herein, refers to a compound selected from —COOH, —C—OH, and

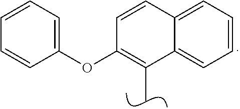 [SMILES: Cc1c(Oc2ccccc2)ccc2ccccc12]

“R¹⁵” as used herein, refers to a compound selected from methyl,

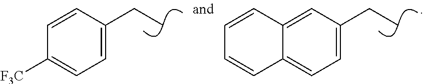

### Compounds of the Invention

In one embodiment, the present invention provides a therapeutic compound of formula A-B-D wherein:
A is selected from —C—OH, —COOH, a compound of formula (I)

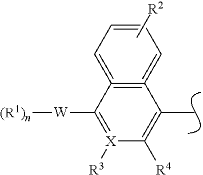

and formula

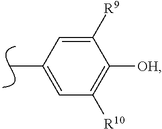

### (II)

wherein:
n is an integer of 0 or 1;
W, R⁹ and R¹⁰ are each independently selected from an H, a halogen, a nitrile, a carbonyl and a nitro group;
X is selected from C, N, O, P, and S;
R¹ and R² are each independently selected from H, dimethylamine, an optionally substituted alkyl, an optionally substituted aryl and an optionally substituted heteroaryl; and
R³ and R⁴ are each independently selected from, H, O, O—CH₃, O—CH₂-aryl, and a C₁-C₆ alkyl, wherein R³ and R⁴ taken together with the atoms to which they are attached optionally form a 6-membered cycloalkyl or heterocycloalkyl,

B is a linker compound selected from

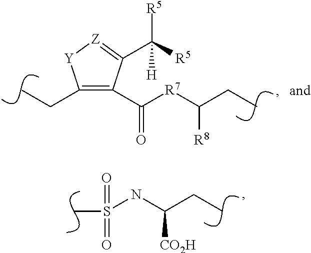

wherein:
Y is C or N;
Z is C or O;
each R⁵ is independently selected from H, an optionally substituted C₂-C₆ alkyl, an optionally

substituted aryl, an optionally substituted heteroaryl and

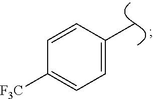

R⁷ is selected from —N—, —NH₂—C₁-C₆ alkyl-NH₂—, and piperazine; and
R⁸ is selected from —CO₂H, and —CO₂—CH₂—CH₃, and
D is a compound of formula (II) wherein R⁹ and R¹⁰ are each independently selected from an H, a halogen, a nitrile, a carbonyl and a nitro group,
or a pharmaceutically acceptable salt, ester or prodrug thereof.

In a preferred embodiment, the present invention provides a therapeutic compound of

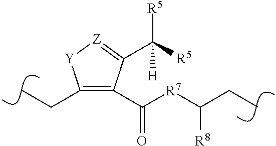

formula A-B-D wherein A is a compound of formula (I), B is and D is a compound of formula (II), or a pharmaceutically acceptable salt, ester or prodrug thereof.

In a more preferred embodiment, the present invention provides a therapeutic compound of formula (III),

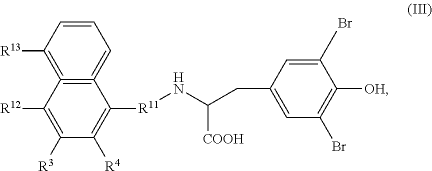

wherein:
R³ and R⁴ are each independently selected from, H, O—CH₃, O—CH₂-phenyl, and a C₆ alkyl, wherein R³ and R⁴ taken together with the atoms to which they are attached optionally form a 6-membered cycloalkyl;

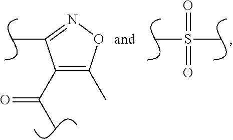

R¹¹ is selected from
R¹² is selected from H and Cl, and
R¹³ is selected from H and dimethylamine.

In an even more preferred embodiment, the present invention provides a therapeutic compound of formula (III) wherein R¹¹ is

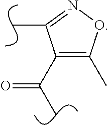

In a yet even more preferred embodiment, the present invention provides a therapeutic compound of formula (III) wherein wherein R¹¹ is

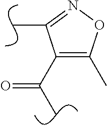

and each of R³, R⁴, R¹² and R¹³ is H.

In another yet even more preferred embodiment, the present invention provides a therapeutic compound of formula (III) wherein wherein R¹¹ is

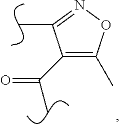

R⁴ is O—CH₃ and each of R³, R¹² and R¹³ is H.

In another yet even more preferred embodiment, the present invention provides a therapeutic compound of formula (III) wherein wherein R¹¹ is

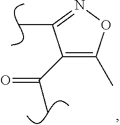

R⁴ is O—CH₂-phenyl, and each of R³, R¹² and R¹³ is H.

In another yet even more preferred embodiment, the present invention provides a therapeutic compound of formula (III) wherein wherein R¹¹ is

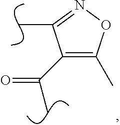

R³ and R⁴ taken together with the atoms to which they are attached form a 6-membered cycloalkyl, R¹² is Cl and R¹³ is H.

In another yet even more preferred embodiment, the present invention provides a therapeutic compound of formula (III) wherein each of R³, R⁴ and R¹² is H, R¹¹ is

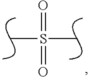

and
R¹³ is dimethylamine.

In a most preferred embodiment, the present invention provides a therapeutic compound selected from

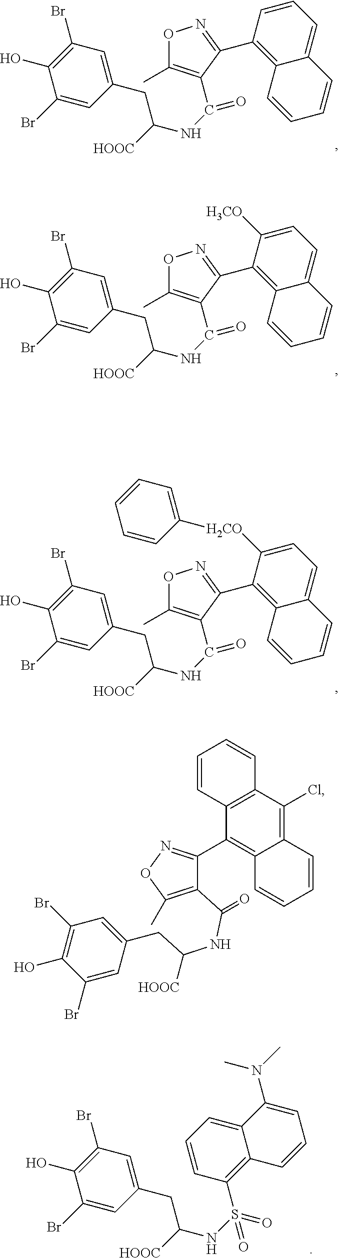

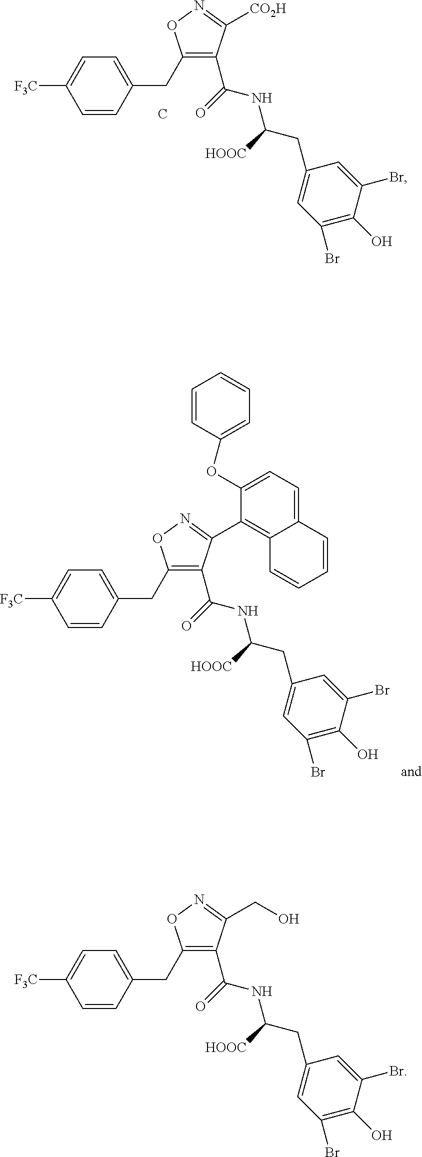

In another embodiment, the present invention provides a therapeutic compound of formula (IV),

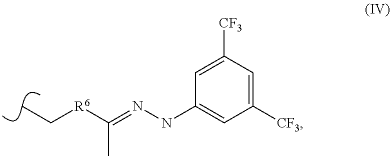

wherein:

R⁶ is selected from

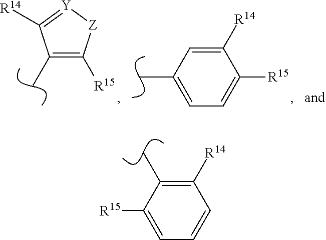

wherein Y is C or N and Z is C or O,

R¹⁴ is selected from —COOH, —C—OH, and and

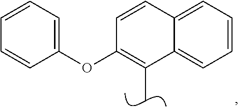 [SMILES: Cc1c(Oc2ccccc2)ccc2ccccc12]

and

R¹⁵ is selected from methyl,

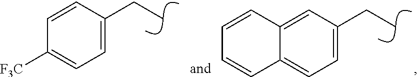

wherein when R¹⁵ is methyl then R¹⁴ is not —COOH or —C—OH.

In another most preferred embodiment, the present invention provides a therapeutic compound selected from

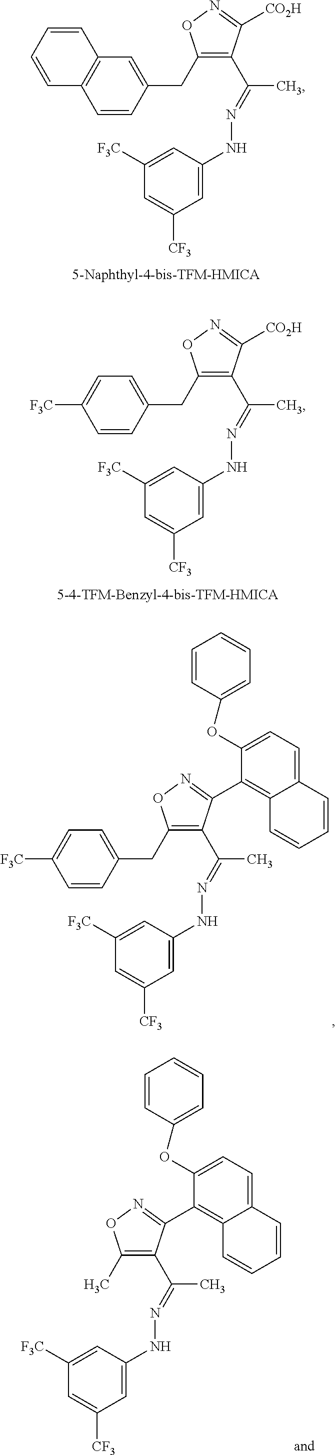 [SMILES: C/C(=N\Nc1cc(C(F)(F)F)cc(C(F)(F)F)c1)c1c(C(=O)O)noc1Cc1ccc2ccccc2c1 ; C/C(=N\Nc1cc(C(F)(F)F)cc(C(F)(F)F)c1)c1c(C(=O)O)noc1Cc1ccc(C(F)(F)F)cc1 ; C/C(=N\Nc1cc(C(F)(F)F)cc(C(F)(F)F)c1)c1c(-c2c(Oc3ccccc3)ccc3ccccc23)noc1Cc1ccc(C(F)(F)F)cc1 ; C/C(=N\Nc1cc(C(F)(F)F)cc(C(F)(F)F)c1)c1c(-c2c(Oc3ccccc3)ccc3ccccc23)noc1C]

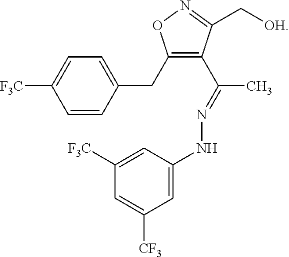 [SMILES: C/C(=N\Nc1cc(C(F)(F)F)cc(C(F)(F)F)c1)c1c(CO)noc1Cc1ccc(C(F)(F)F)cc1]

### Compositions of the Invention

The present invention also provides pharmaceutical compositions that comprise compounds of the present invention formulated together with one or more pharmaceutically acceptable carriers. The pharmaceutical compositions can be specially formulated for oral administration in solid or liquid form, for parenteral administration or for rectal administration.

The pharmaceutical compositions of this invention can be administered to humans and other mammals orally, rectally, parenterally, intracisternally, intravaginally, transdermally (e.g. using a patch), transmucosally, sublingually, pulmonary, intraperitoneally, topically (as by powders, ointments or drops), bucally or as an oral or nasal spray. The terms “parental” or “parenterally,” as used herein, refers to modes of administration which include intravenous, intramuscular, intraperitoneal, intrasternal, subcutaneous and intraarticular injection and infusion.

In another aspect, the present invention provides a pharmaceutical composition comprising a component of the present invention and a physiologically tolerable diluent. The present invention includes one or more compounds as described above formulated into compositions together with one or more physiologically tolerable or acceptable diluents, carriers, adjuvants or vehicles that are collectively referred to herein as diluents, for parenteral injection, for intranasal delivery, for oral administration in solid or liquid form, for rectal or topical administration, among others.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof.

These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol, glycerol monostearate, and PEG caprylic/capric glycerides ; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology,Volume XIV, Academic Press, New York, N.Y. (1976), p. 33 et seq. Diseases to be Treated

System x_{c}⁻ (“Sx_{c}⁻”) is implicated in many diseases including tumors including cancer including brain tumor growth, chemosensitivity, chemoresistance, seizures, schizophrenia, Parkinson's, and viral infections of the brain. Cancers in which Sx_{c}⁻ has been shown to be over-expressed include but are not limited to glioblastomas, triple negative breast cancer and bladder cancer. Potent and selective inhibition of Sx_{c}⁻ is an important factor in treating these diseases.

### EXAMPLES

### Example 1

### Syntheses

### General Synthesis Strategy for Compounds Containing a 4, 5 Isoxazole Linker

The novel analogues reported in this study were prepared from the bromo acetal (6) shown in Scheme 1 (Nelson et al., The catalytic asymmetric addition of alkyl- and aryl-zinc reagents to an isoxazole aldehyde, Tetrahedron Lett, 2008 Oct 6, 49(41), 5957-5960).

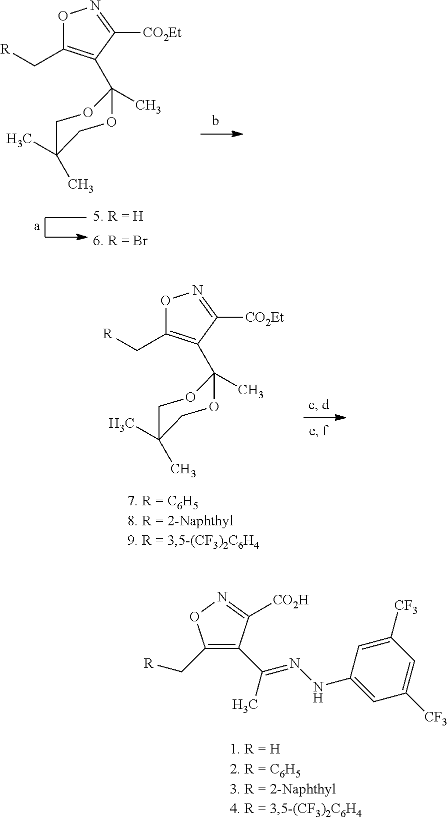

Suzuki-Miyaura palladium (McDaniel et al., Suzuki-Miyaura Cross-Coupling of Benzylic Bromides Under Microwave Conditions, Tetrahedron Lett, 2011 October 26, 52(43), 5656-5658.) catalyzed couplingwith the corresponding arylboronic acids put the C-5 aryl in place, (7-9), hydrolysis of the acetal, hydrazone condensation (Patel et al., Isoxazole analogues bind the system xc-transporter: structure-activity relationship and pharmacophore model, Bioorg Med Chem, 2010 Jan 1, 18(1), 202-13), and hydrolysis of the C-3 ester under basic conditions to arriveat the products (2-4) was then accomplished as previously described (Matti et al., Microwave accelerated synthesis of isoxazole hydrazide inhibitors of the Sx_{c}⁻ transporter: initial homology model, Bioorg Med Chem Lett 2013 Nov 1, 23(21), 5931-5). To enhance solubility dimethyl sulfoxide (“DMSO”) was included in the preparation of stock solutions of the inhibitors. The concentration of DMSO present following dilution into the assay solutions was 60.5% vol./vol. Previous studies confirmed that this amount of DMSO had no effect on transport rates.

General Synthesis Strategy for Compounds containing Hindered 3-aryl-isoxazole-DBT Ligands

All reactions were performed under inert atmosphere. Chemicals were purchased from TCI or Aldrich Chemical Company, all commercial reagents are routinely examined for purity by NMR and TLC, and recrystallized or distilled as appropriate. Solvents were reagent grade. Tetrahydrofuran (“THF”) was dried over sodium/benzophenone and distilled prior to use. Triethylamine (“TEA”) was dried with calcium hydride (“CaH₂”). Melting points were determined in open capillary tubes on a Melt-Temp apparatus and are uncorrected. NMR spectra were obtained using either a Varian 400 MHz Unity Plus or a Varian NMR systems 500 MHz spectrometer, in deuteriochloroform unless otherwise noted. Infrared spectra were obtained on a thermo-Nicolet 633 FT-IR spectrometer.

Chemical shifts (δ) are reported using CHCl₃ (7.26 ppm for ¹H), CDCl₃ (77 ppm for ¹³C) as references. High resolution mass spectra (HRMS) were obtained using a Micromass electrospray ionization (ESI)/time-of-flight mass spectrometry (LCTOF). Mass spectrometer samples were introduced using a Waters model 2690 separations module HPLC fitted with a C-18 reversed phase column (2.1 mm i.d., 5 cm). Elemental analyses for C, H, and N were performed by Midwest Microlab, Indianapolis, Ind. All reactions were monitored by Thin Layer Chromatography (TLC). Purification was performed by flash column chromatography, and analytical samples were prepared by PTLC. Analytical LCMS (UV at 254 nm) and NMR were used to establish the purity of targeted compounds. All compounds that were evaluated in biochemical and biophysical assays had >95% purity as determined by ¹H NMR and LCMS.

Dibromotyrosine (“DBT”) was prepared as previously described. Ding W. et al., The synthesis, distribution, and anti-hepatic cancer activity of YSL, Bioorg Med Chem, Sep. 15, 2004, 12(18), 4989-4994. The nitrile oxide cycloaddition procedure was used to prepare the sterically hindered isoxazoles has been described previously. Mirzaei Y. R. et al., Improved synthesis of 3-aryl isoxazoles containing fused aromatic rings. Tetrahedron, Dec. 16, 2012, 68 (50), 10360-10364.

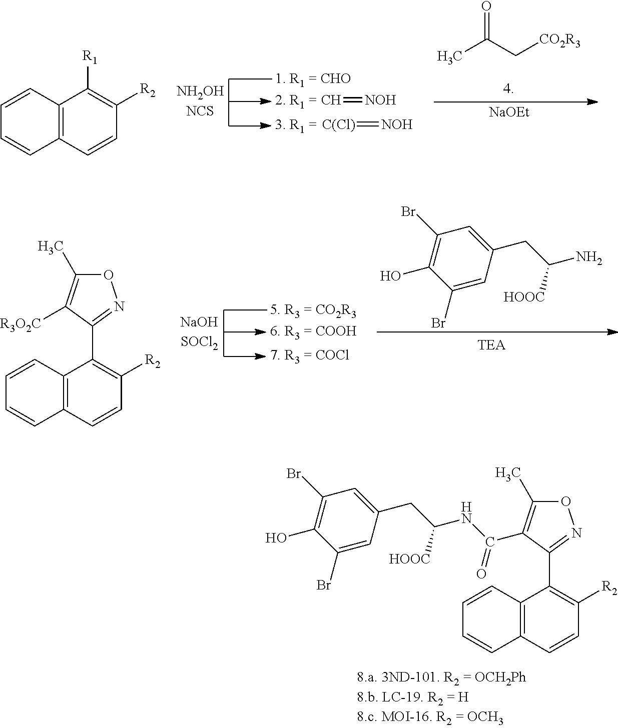

The general synthesis of DBT analogs begins with the aryl aldehydes (1) which are treated with hydroxylamine to produced the corresponding oximes (2), followed by treatment with N-Chloro succinimide (NCS) in carbon tetrachloride or chloroform to produce the oximidoyl chloride (3). Nitrile oxide cycloaddition with the sodium salt of a ketoester provides the 3-aryl isoxazole (5). Hydrolysis to the carboxylic acid (6) and transformation to the acyl chloride with thionyl chloride provided the acid chloride (7), upon which reaction with dibromotyrosine (DBT, Ding 2004) produced the 3-aryl isoxazole DBT ligands (8).

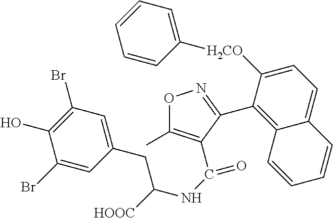

### Preparation of (2S)-2-(3-(2-(benzyloxy)naphthalen-1-yl)-5-methylisoxazole-4-carboxamido)-3-(3,5-dibromo-4-hydroxyphenyl)propanoic acid, 8a (3ND-101)

2-(Benzyloxy)-1-naphthaldehyde 1 (1.000 g, 3.8124 mmol), hydroxylamine hydrochloride (0.5298 g), and sodium acetate.3H₂0 (1.5564 g) was dissolved in THF/ethanol/water (20 mL: 10 mL: 10 mL). After stirring at rt for overnight, the mixture was concentrated then washed 4×50 H₂O, 2×75 mL Brine and 2×25 mL EtOAc, dried over anhydrous sodium sulfate, filtered, and concentrated to produce the oxime 2, 1.057 g (95%). The oxime 2 (1.0065 g, 3.6294 mmol) was treated with N-Chlorosuccinimide (0.5463 g), pyridine (3 drops) in 40 mL chloroform was stirred at room temperature for 5 hours. The solution was washed with 3×50 mL H₂O, 2×50 mL Brine, and 2×25 mL chloroform, then dried over anhydrous sodium sulfate, filtered, and concentrated to produce the product 3. To a solution of the nitrile oxide 3 in ethanol (35 mL), was added ethyl acetoacetate (lmL) and sodium (0.150 g) in ethanol (100 mL), dropwise, and the reaction mixture allowed to stir at room temperature overnight. The solution was concentrated, washed with 2×75 mL H₂O, 2×50 mL brine, then dried over anhydrous sodium sulfate, filtered, and concentrated. Product 5 was collected, 1.3959 g, 99%. Ester 5 (1.0098 g, 3.629 mmol) in methanol/THF (l5 mL:l5 mL) was refluxed (90° C.) in 2 M KOH for 3 h then allowed to cool to rt overnight, acidified with 1N aqueous HCl, to give the carboxylic acid 6 (1.2781 g, 98%). The carboxylic acid 6 was stir in an ice bath and allowed to warm up overnight in neat SOCl₂ (25 mL), the mixture was then concentrated using hexanes, then dry dichloromethane three times and the residue was used without further purification in the next step. To acid chloride 7 in 60 mL of DCM was added (S)-2-amino-3-(3,5-dibromo-4-hydroxyphenyl)propanoic acid¹ (0.9909 g) and 2 mL TEA, the mixture was stirred at rt for 24 hours, after which time it was concentrated and purified by flash chromatography using 4:1 EtOAc:MeOH to give the product 8a (1.4221 g, 89%). ¹H NMR (400 MHz, Methanol-d₄) δ ppm 7.96 (d, J=9.03 Hz, 1H), 7.82 (d, J=7.78 Hz, 1H), 7.31 (m, 10H), 6.94 (br. s., 2H), 5.18 (br. s., 2H), 4.30 (t, J=5.58 Hz, 1H), 2.90 (m, 1H), 2.72 (s, 2H), 2.55 (dd, J=14.18, 6.27 Hz, 1H). ¹³C NMR (101 MHz, Methanol-d₄) δ ppm 177.48, 162.97, 158.29, 156.30, 150.83, 138.41, 134.53, 133.86, 133.59, 132.91, 130.54, 129.67, 129.44, 129.04, 128.88, 128.42, 125.53, 124.72, 115.94, 111.96, 111.58, 72.25, 56.35, 37.24, 13.14. Mass Spectrum for C₃₁H₂₄Br₂N₂O₆ 679.0139 (M+1, ⁷⁹Br₂, 50% rel. intensity); 681.0175, (M+1, ⁷⁹Br⁸¹Br, 100); 683.0147 (⁸¹Br₂, 53%).

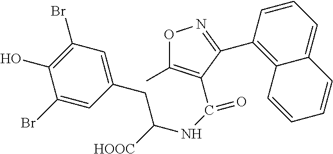 [SMILES: Cc1onc(-c2cccc3ccccc23)c1C(=O)NC(Cc1cc(Br)c(O)c(Br)c1)C(=O)O]

### 3-(3,5-dibromo-4-hydroxyphenyl)-2-(5-methyl-3-(naphthalen-1-yl)isoxazole-4-carboxamido)propanoic acid. (8. b. LC-19)

Mass Spectrum for C₂₄H₁₈Br₂N₂O₆ 572.9881 (M+1, ⁷⁹Br₂, 50% rel. intensity); 574.9929, (M+1, ⁷⁹Br⁸¹Br, 100); 576.9908 (⁸¹Br₂, 53%).

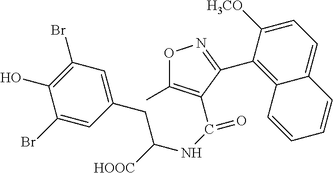 [SMILES: COc1ccc2ccccc2c1-c1noc(C)c1C(=O)NC(Cc1cc(Br)c(O)c(Br)c1)C(=O)O]

### 3-(3,5-dibromo-4-hydroxyphenyl)-2-(5-methyl-3-(2-methoxynaphthalen-1-yl)isoxazole-4-carboxamido)propanoic acid (8.c. MOI-16)

The above compound C₂₅H₂₀Br₂N₂O₆ (MOI-16) has the (S)-absolute configuration at the alpha amido carboxylic acid position, and crystallizes in exclusively the (R)-configuration at the chiral axis joining the isoxazole and naphthalene rings. There is an intermolecular halogen bond between Brl and C l′, which is within both the distance (3.3448 Å), and dihedral angle (139.67°) range. Two additional hydrogen bonds connect MOI-16 in the unit cell: between the isoxazole N1 and the Phenol moiety H6′-O6′of the dibromotyrosine (DBT) of 2,859 Å, and between the Carboxylic acid O4-H4 and the carbonyl O3′ of the trans amide of 2.590A. The dihedral angle between the mean plane of the isoxazole and naphthalene rings approaches orthogonal at 88.99°, while the isoxazole and dibromotyrosine mean planes are roughly parallel with a dihedral angle of 20.43°. These close contacts are expected to be analogous to those found in the drug-receptor interaction of the title compound and the System Xc-antiporter, of which it is a potent inhibitor.

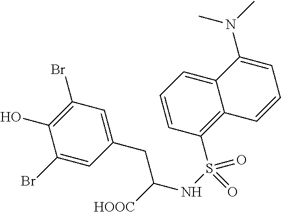 [SMILES: CN(C)c1cccc2c(S(=O)(=O)NC(Cc3cc(Br)c(O)c(Br)c3)C(=O)O)cccc12]

### Preparation of 3-(3,5-dibromo-4-hydroxyphenyl)-2-(5-(dimethylamino)naphthalene-1-sulfonamido)propanoic acid (danzyl-DBT)

To dansyl chloride (Aldrich, 100 mg, 0.37 mmol) in 10 ml methylene chloride and 1 ml of TEA was added 3,5-DBT hydrobromide (156 mg, 0.37 mmol), the mixture was stirred for 24 h at R.T. All volatiles were evaporated and the residue was dissolved in water. Three equivalents of NaOH were added and the mixture was applied on a plug of cellulose cation exchanger (Sigma-Aldrich). Elution with water and methanol yielded 113 mg of the pure product, 53%.

¹H NMR (500 MHz, d₆-acetone) δ ppm 8.74 (d, J=8.07 Hz, 1H) 8.48-8.58 (m, 1H) 8.15-8.24 (m, 1H) 8.07-8.13 (m, 1H) 7.45-7.56 (m, 2H) 7.19-7.27 (m, 1H) 7.13 (s, 2H) 4.08 (td, J=9.29, 4.65 Hz, 1H) 3.20 (dd, J=14.67, 7.34 Hz, 1H) 2.94 (dd, J=14.18, 13.45 Hz, 1H) 2.89 (s, 6H). ¹³C NMR (126 MHz, d₆-acetone) δ ppm 173.06; 150.41; 144.65; 137.04; 134.23 /(s, 1 C) 132.12 (s, 1 C) 131.44 (s, 1 C) 130.97 (s, 1 C) 130.59 (s, 1 C) 130.12 (s, 1 C) 128.97 (s, 1 C) 124.26 (s, 1 C) 120.83 (s, 1 C) 116.59; 111.22; 58.59; 46.17; 37.73. [a]_{D}-50.5 (c 3.7, EtOH).

Accurate Mass calculated for C₂₁H₂₃N₂O₅SBr₂: m/Z 572.9694, found: 572.9650. Error: −7.8 ppm.

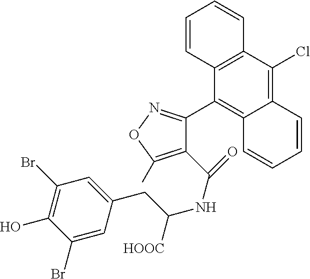 [SMILES: Cc1onc(-c2c3ccccc3c(Cl)c3ccccc23)c1C(=O)NC(Cc1cc(Br)c(O)c(Br)c1)C(=O)O]

### Preparation of 2-(3-(10-chloroanthracen-9-yl)-5-methylisoxazole-4-carboxamido)-3-(3,5-dibromo-4-hydroxyphenyl)propanoic acid (AIM-DBT)

Under an argon atmosphere, 89 mg of the acyl chloride, 85 mg of 3, 5-DBT, 1 ml TEA and 7 ml methylene chloride were combined at R.T. and stirred for 20 h. Next, the volatiles were evaporated under reduced pressure; the residue was acidified with HCl, extracted 3×20 ml AcOEt, dried (sodium sulfate) and separated on a silica preparative plate (twice) with AcOEt/Hex/MeOH (12:12:1), followed by high vacuum pumping for 3 days. Isolated yield: 89 mg, 54%.

¹H NMR (500 MHz, CDCl₃) δ ppm 8.45-8.53 (m, 2H) 7.52-7.62 (m, 4H) 7.44-7.50 (m, 4H) 6.73 (s, 2H) 5.27 (d, J=6.85 Hz, 1H) 4.35 (q, J=6.03 Hz, 1H) 2.97 (s, 3H) 2.73 (dd, J=14.43, 5.87 Hz, 1H) 2.32 (dd, J=14.43, 5.87 Hz, 1H)

¹³C NMR (126 MHz, CDCl₃) δ ppm 176.31 (s, 1 C) 173.71 (s, 1 C) 160.36 (s, 1 C) 157.53 (s, 1 C) 148.24 (s, 1 C) 132.64 (s, 1 C) 131.95 (s, 2 C) 131.24 (s, 1 C) 130.89 (s, 1 C) 129.73 (s, 1 C) 128.52 (s, 1 C) 128.38 (s, 1 C) 127.81 (s, 1 C) 127.66 (s, 1 C) 127.13 (s, 1 C) 127.11 (s, 1 C) 125.22 (s, 1 C) 125.18 (s, 2 C) 124.55 (s, 1 C) 119.72 (s, 1 C) 112.22 (s, 1 C) 109.64 (s, 2 C) 52.35 (s, 1 C) 35.34 (s, 1 C) 14.16. [c]p -58.823 (acetone).

Accurate Mass calculated for C₂₈H₂₀N₂O₅³⁵Cl⁷⁹Br⁸¹Br (M+1)′: m/Z: 658.9407, found: 658.9346. Error: 9.3 ppm.

### Example 2

### Studies

### Inhibition of System X_{c}⁻

### Cell Culture

SNB-19 glioma cells, purchased from American Type Culture Collection (Manassas, Va.), were grown in DMEM/F-12 medium (pH 7.4) containing 1 mM pyruvate and 16 mM NaHCO₃ and supplemented with 10% fetal calf serum. The cells were cultured in 150 cm² flasks (Corning) and maintained at 37° C. in a humidified atmosphere of 5% CO₂. In the ³H-L-Glu uptake experiments, cells were seeded in 12 well culture plates (Costar) at a density of 5×10⁴ cells/well and maintained for 3 days until 80-90% confluent. Protein concentrations were determined by the bicinchoninic acid (BCA) method (Pierce).

### Glutamate Uptake Assay

Uptake of ³H-L-Glu into cultured cells was quantified using a modification of the procedure of Martin and Shane as previously described by Patel et al. (supra). Briefly, after removal of culture media, wells were rinsed three times and pre-incubated in 1 ml Na⁺-free HEPES buffered (pH 7.4) Hank's balanced salt solution (HBHS) at 30° C. for 5 min. The Nat free buffer contained: 137.5 mM choline Cl, 5.36 mM KCl, 0.77 mM KH₂PO₄, 0.71 mM MgSO⁴.7H₂O, 1.1 mM CaCl₂, 10 mM D-glucose, and 10 mM HEPES. Uptake was initiated by aspiration of the pre-incubation buffer and the addition of a 500 gl aliquot of Na⁺-free transport buffer containing ³H-L-Glu (4-16 mCi/m1) mixed with L-Glu (10-500 μM, final concentration). In those assays that evaluated inhibitor activity, the 500 gl aliquot of transport buffer contained both the ³H-L-Glu and potential inhibitors to ensure simultaneous addition. Following a 5 min incubation at 30° C., the assays were terminated by three sequential 1 ml washes with ice cold buffer after which the cells were dissolved in 1 ml of 0.4 M NaOH for 24 h. An aliquot (200 μl) was then transferred into a 5 ml glass scintillation vial and neutralized with the addition of 5 gl glacial acetic acid followed by 3.5 ml Liquiscint© scintillation fluid (National Diagnostics) to each sample. Incorporation of radioactivity was quantified by liquid scintillation counting (LSC, Beckman LS 6500). Values are reported as mean±S.E.M. and are corrected for non-specific uptake (e.g., leakage and binding) by subtracting the amount of ³H-L-Glu accumulation at 4° C.

### Results

The inhibitory activity of the compounds was determined by quantifying the ability of the analogues to reduce the accumulation of ³H-L-Glu into human SNB-19 glioblastoma cells under Cl-dependent (Na-free) conditions. A number of glioma cell lines, including SNB-19, express markedly higher levels of Sx_{c}⁻ and reduced levels of the sodium-dependent excitatory amino acid transporters (“EAATs”) than do primary astrocytes, making them well suited for pharmacological assays (Ye et al., Compromised glutamate transport in human glioma cells: reduction-mislocalization of sodium-dependent glutamate transporters and enhanced activity of cystine-glutamate exchange, J Neurosci, 1999 Dec. 15, 19(24), 10767-77). The compounds of the invention were initially screened at a single concentration of substrate (100 μM ³H-L-Glu) and isoxazole (500 μM) to confirm inhibitory activity. As reported in Table 1, the analogues almost completely blocked the uptake of the ³H-LGlu into the cells under these conditions. (The data are reported as % of control uptake, thus the smaller the number the greater the level of inhibition.)

TABLE 1
Inhibition of Glutamate Uptake by Compounds of the Invention

| | | |
|---|---|---|
| Compound | ³H-L-Glu | K_{I} ³H-L-Glu |
| (250 μM) | Uptake (% control) | Uptake (μM) |

| | | | |
|---|---|---|---|
| 3ND-101 | 8 | (100 μM) | 100 |
| | 1 | (500 μM) | |
| LC-19 | 23 | (100 μM) | 100 |
| | 6 | (500 μM) | |
| MOI-16 | 32 | (100 μM) | 100 |
| | 8 | (500 μM) | |
| Danzyl-DBT | 52 | (100 μM) | 25 |
| AIM-DBT | 8 | (100 μM) | 15 |
| 5-4-TFM-Benzyl-4- | 14 | (100 μM) | |
| bis-TFM-HMICA | | | |
| 5-Naphthyl-4-bis- | 6 | (100 μM) | |
| TFM-HMICA | | | |

As can be seen in Table 1, compounds 3ND-101 and AIM-DBT had the greatest inhibitory activity. This suggests that larger lipophilic groups joined by an isoxazole linker may be more effective inhibitors of Sx_{c}⁻.

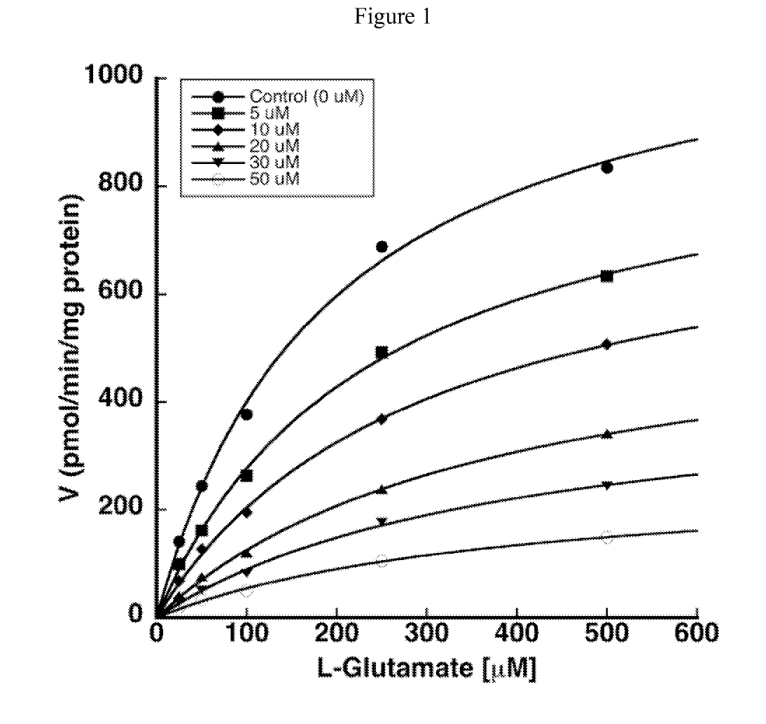

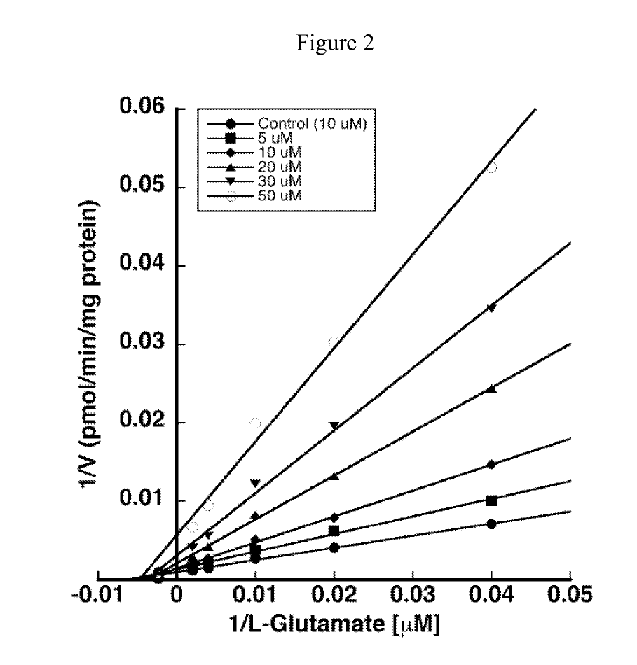

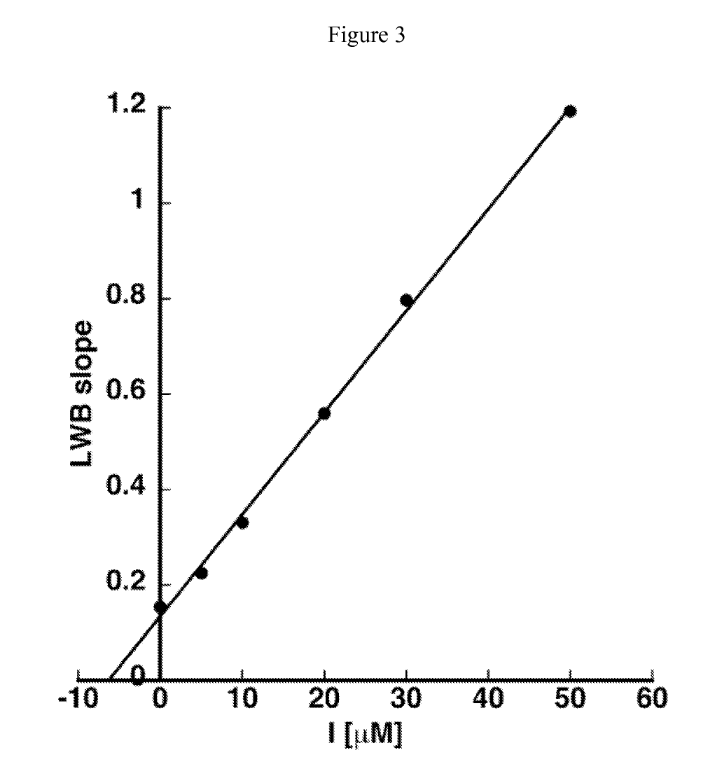

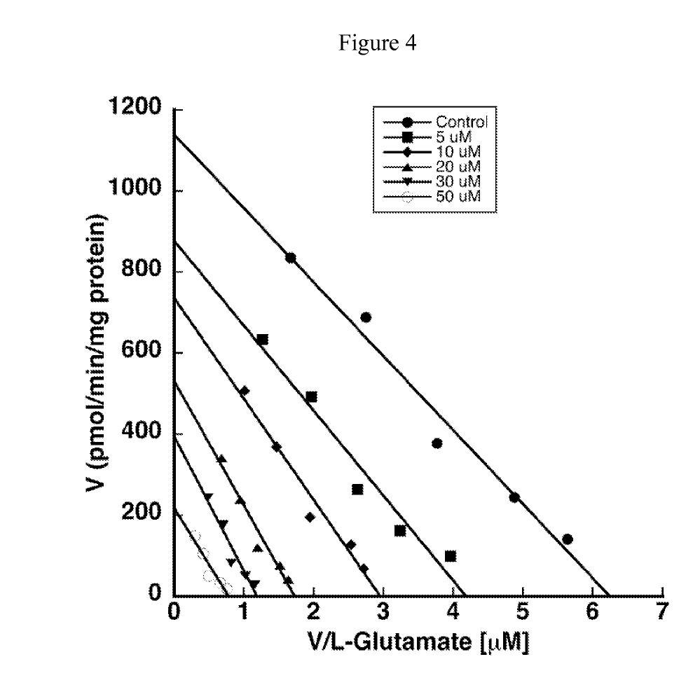

Further, as demonstrated in FIGS. 1-4 for 5-4-TFM-Benzyl-4-bis-TFM-HMICA di-substituted analogues exhibited a pattern of inhibition consistent with noncompetitive inhibition. Both the V vs. S (FIG. 1) and LWB plots (FIG. 2) demonstrate that the inhibitors produced a decrease in V. with little or no change in Kₘ, as would be expected of noncompetitive inhibitors. A slope replot for 5-4-TFM-Benzyl-4-bis-TFM-HMICA is included in FIG. 3, (average Kᵢ=3±1 μM, n=5). Competitive and noncompetitive inhibitors can also be distinguished by replots from LWB graphs of either K_{m,apparent} vs. [I], linear for competitive mechanisms, or I/V_{max,apparent} vs. [I], linear for noncompetitive inhibition. In the instance of 5-4-TFM-Benzyl-4-bis-TFM-HMICA the replot of I/V_{max,apparent} vs. [I] was indeed linear and yielded a Kᵢ of about 8 μM (plots not shown). If both the slope replot and the I/V_{max apparent} vs. [I] replot max apparent vs. yield similar Kᵢ values, as is the case for 5-4-TFM-Benzyl-4-bis-TFM-HMICA, the analogue is considered to be acting as a “pure” noncompetitive inhibitor, where the binding of the compound does not alter the binding affinity of the substrate. As the identification of the di-substituted isoxazoles as noncompetitive inhibitors was unexpected, the kinetic data was also analyzed using the Eadie-Hofstee method as a second graphical approach. As depicted in FIG. 4 for 5-4-TFM-Benzyl-4-bis-TFM-HMICA, the plots of V vs. V/[S] yielded a series of parallel lines, a pattern indicative of noncompetitive inhibition.

What is claimed is:

1. A compound of formula A-B-D wherein:
A is selected from —C—OH, —COOH, a compound of formula (I) 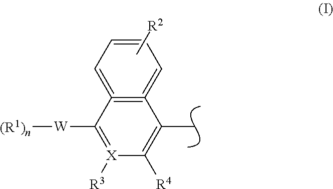
and formula (II) 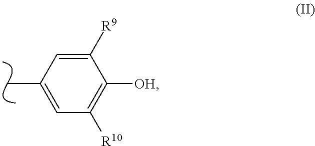
wherein:
n is an integer of 0 or 1;
W, R⁹ and R¹⁰ are each independently selected from an H, a halogen, a nitrile, a carbonyl and a nitro group;
X is selected from C, N, O, P, and S;
R¹ and R² are each independently selected from H, dimethylamine, an optionally substituted alkyl, an optionally substituted aryl and an optionally substituted heteroaryl; and
R³ and R⁴ are each independently selected from, H, O, O—CH₃, O—CH₂-aryl, and a C₁-C₆ alkyl, wherein R³ and R⁴ taken together with the atoms to which they are attached optionally form a 6-membered cycloalkyl or heterocycloalkyl,
B is a linker compound selected from 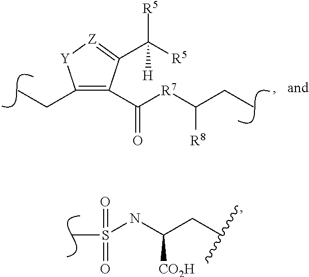
wherein:
Y is C or N;
Z is C or 0;
each R⁵ is independently selected from H, an optionally substituted C₂-C₆ alkyl, an optionally substituted aryl, an optionally substituted heteroaryl and 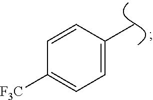
R⁷ is selected from —N—, —NH₂—C₁-C₆ alkyl-NH₂—, and piperazine; and
R⁸ is selected from —CO₂H, and —CO₂—CH₂—CH₃, and
D is a compound of formula (II) wherein R⁹ and R¹⁰ are each independently selected from and H, a halogen, a nitrile, a carbonyl and a nitro group,
or a pharmaceutically acceptable salt, ester or prodrug thereof.

2. The compound of claim 1 wherein A is a compound of formula (I) and B is

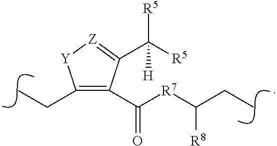

3. A compound of formula (III), 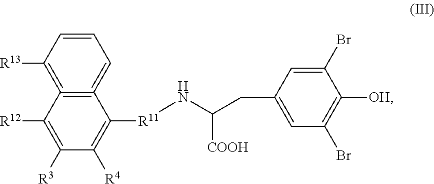
wherein:
R³ and R⁴ are each independently selected from, H, O—CH₃, O—CH₂-phenyl, and a C₆ alkyl, wherein R³ and R⁴ taken together with the atoms to which they are attached optionally form a 6-membered cycloalkyl; 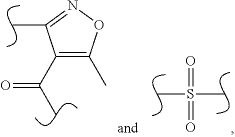
R¹¹ is selected from
R¹² is selected from H and Cl, and
R¹³ is selected from H and dimethylamine.

4. The compound of claim 3, wherein R¹¹ is

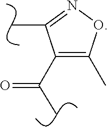

5. The compound of claim 4, wherein each of R³, R⁴, R¹² and R¹³ is H.

6. The compound of claim 4, wherein:
R⁴ is O—CH₃; and
each of R³, R¹² and R¹³ is H.

7. The compound of claim 4, wherein:
R⁴ is O—CH₂-phenyl; and
each of R³, R¹² and R¹³ is H.

8. The compound of claim 4, wherein:
R³ and R⁴ taken together with the atoms to which they are attached form a 6-membered cycloalkyl;
R¹² is Cl; and
R¹³ is H.

9. The compound of claim 3, wherein:
each of R³, R⁴ and R¹² is H;
R¹¹ is 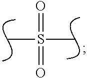
and
R¹³ is dimethylamine.

10. The compound of claim 1 selected from

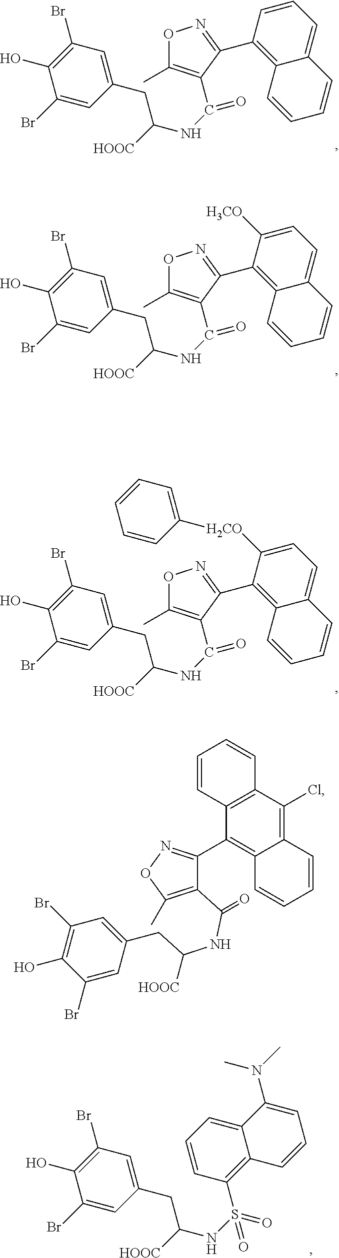

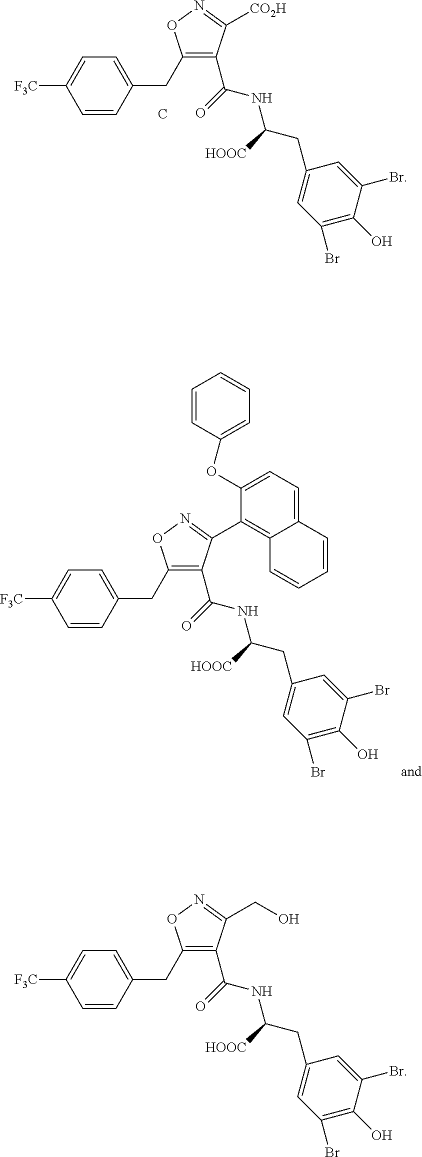

11. A compound of formula (IV), 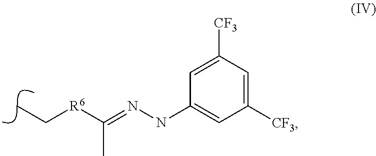
wherein:
R⁶ is selected from 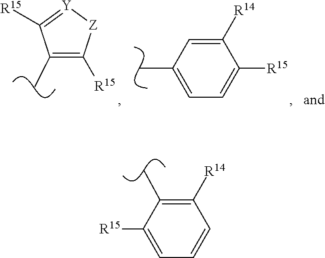
wherein Y is C or N and Z is C or O,
R¹⁴ is selected from —COOH, —C—OH, and and 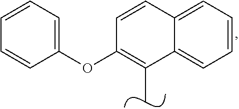 [SMILES: Cc1c(Oc2ccccc2)ccc2ccccc12]
R¹⁵ is selected from methyl, 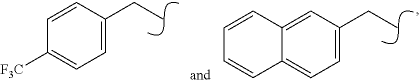
wherein when R¹⁵ is methyl then R¹⁴ is not —COOH or —C—OH.

12. The compound of claim 11 wherein R⁶ is

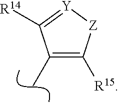

13. The compound of claim 12 wherein R¹⁴ is 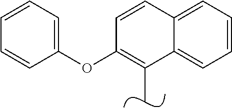 [SMILES: Cc1c(Oc2ccccc2)ccc2ccccc12]
R¹⁵ is methyl.

14. The compound of claim 12 wherein R¹⁴ is —C—OH and R¹⁵ is

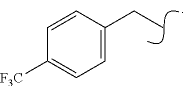

15. The compound of claim 11 selected from

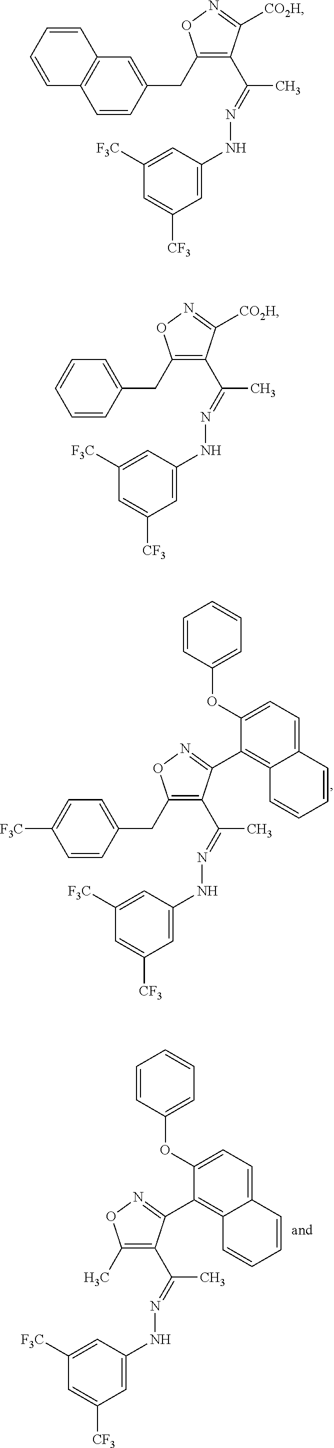 [SMILES: C/C(=N\Nc1cc(C(F)(F)F)cc(C(F)(F)F)c1)c1c(C(=O)O)noc1Cc1ccc2ccccc2c1 ; C/C(=N\Nc1cc(C(F)(F)F)cc(C(F)(F)F)c1)c1c(C(=O)O)noc1Cc1ccccc1 ; C/C(=N\Nc1cc(C(F)(F)F)cc(C(F)(F)F)c1)c1c(-c2c(Oc3ccccc3)ccc3ccccc23)noc1Cc1ccc(C(F)(F)F)cc1 ; C/C(=N\Nc1cc(C(F)(F)F)cc(C(F)(F)F)c1)c1c(-c2c(Oc3ccccc3)ccc3ccccc23)noc1C]

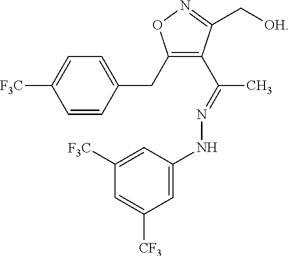 [SMILES: C/C(=N\Nc1cc(C(F)(F)F)cc(C(F)(F)F)c1)c1c(CO)noc1Cc1ccc(C(F)(F)F)cc1]

16. A composition comprising a compound of claim 1 and one or more pharmaceutically acceptable carriers.

17. A method of treating a tumor comprising administering to a subject in need thereof a therapeutically effective amount of a compound of claim 1.

18. The method of claim 17 wherein the tumor is a glioblastoma multiforme.

19. The method of claim 17 wherein the tumor is triple negative breast cancer.

20. A method of treating a seizure comprising administering to a subject in need thereof a therapeutically effective amount of a compound of claim 1.

21. The method of claim 20 wherein the seizure is an epileptic seizure.

22. The method of claim 20 wherein the seizure is status epilepticus.

23. A method for detecting cancer in vivo, comprising:
(i) administering to a patient in need thereof a diagnostically effective amount of a compound of claim 1 wherein at least one atom is an atom selected from carbon-11 (¹¹C), fluorine-18 (¹⁸F), nitrogen-13 (¹³N), oxygen-15 (¹⁵O) or a combination thereof;
(ii) detecting whether a tissue suspected of having cancer in the patient retains a higher level of the compound of (i) than surrounding tissue,
wherein a higher retention level of the compound of (i) indicates cancer and wherein the detection is carried out by positron emission tomography (PET) scanning

24. A method for detecting cancer in vivo, comprising:
(i) administering to a patient in need thereof a diagnostically effective amount of a compound of claim 1;
(ii) detecting whether a tissue suspected of having cancer in the patient retains a higher level of the compound of (i) than surrounding tissue,
wherein a higher retention level of the compound of (i) indicates cancer and wherein the detection is carried out by fluorescent imaging.
